(19) United States
(12) Patent Application Publication
(10) Pub. No.: US 2014/0018705 A1
(43) Pub. Date: Jan. 16, 2014

(54) GAIT ANALYSIS METHOD AND GAIT ANALYSIS SYSTEM

(71) Applicant: National Cheng Kung University, Tainan City (TW)

(72) Inventors: Jeen-Shing WANG, Tainan City (TW); Che-Wei LIN, Ji' an Township (TW)

(21) Appl. No.: 13/935,898

(22) Filed: Jul. 5, 2013

Related U.S. Application Data
(60) Provisional application No. 61/668,676, filed on Jul. 6, 2012.

(51) Int. Cl. A61B 5/11; A61B 5/00

(52) U.S. Cl. CPC A61B 5/112; A61B 5/72
USPC 600/595

(57) ABSTRACT

A gait analysis method is implemented by a gait analysis system including a sensing unit, a storing unit storing a plurality of computing programs, and a processing unit electrically connected to the sensing unit and the storing unit, and comprises steps of: sensing a gait by the sensing unit to output a sensing signal, wherein a gait cycle includes a stance phase, a push-off phase, a swing phase and a heel-strike phase; obtaining a signal vector magnitude (SVM) and a signal magnitude subtraction (SMS) by the processing unit according to the sensing signal; identifying the stance phase, push-off phase, swing phase and heel-strike phase according to the SVM and SMS, wherein the push-off phase, swing phase and heel-strike phase are determined according to a dynamic threshold; and implementing a classification of the gait according to the stance phase, push-off phase, swing phase and heel-strike phase.

### CROSS REFERENCE TO RELATED APPLICATIONS

The non-provisional patent application claims priority to U.S. provisional patent application with Ser. No. 61/668,676 filed on Jul. 6, 2012. This and all other extrinsic materials discussed herein are incorporated by reference in their entirety.

### BACKGROUND OF THE INVENTION

1. Field of Invention

The invention relates to a gait analysis method and a gait analysis system.

2. Related Art

Generally, neuropathy and diseases of the musculoskeletal system will cause the problem of walking. From a clinical viewpoint, a disease changes the coordination, harmonization and interaction of the muscle, skeleton, nerves and even joints, and thus influences the patient's gait. In this case, a gait analysis can help figuring out the whole situation. Therefore, the study of using the gait analysis has been developed considerably to solve the clinical problem in the fields of orthopedics, rehabilitation research and neurology.

The gait analysis is mainly for providing a doctor with the detailed assessment data to clarify the crucial problem of the nervous, muscular and skeletal system of the testee. With the assessment, the doctor can make the best plan to practice the treatment, such as surgery, rehabilitation or wearing auxiliary instruments, for the patient. Besides, the gait analysis after the treatment also can help the confirmation of the treatment effect and help the doctor bring up a further plan for improvement. Taking the treatment of orthopedics as an example, the gait analysis can be used as the investigation and assessment both before and after the treatment. Taking the treatment of rehabilitation as an example, the gait analysis can be used as the reference for the diagnosis, assessment and recovery condition. For prosthesis, the gait analysis can be used as the assistance in designing and testing an artificial limb or auxiliary instruments. For neurology, the gait analysis can be used to figure out the movement of a specific limb of the testee for analyzing the characteristics of Parkinson's disease and tracking the treatment effect.

Moreover, in the aspects of preventive medicine and epidemiology, “fall-down” has achieved the third place of the danger to the elderly. By the gait analysis, the factors resulting in the fall-down can be found out. Then, through the early elimination of these factors in cooperation with the patient instructions and training for the elderly, walking assistants or medical auxiliaries, the probability of falling down for the elderly will be decreased to the least, and thus the family and social burdens can be reduced a lot.

### SUMMARY OF THE INVENTION

An objective of the invention is to provide a gait analysis method and a gait analysis system that can analyze and identify the gait of the testee and thus help the doctor provide the medical and healthy instructions for the testee.

To achieve the above objective, a gait analysis method according to the invention is implemented by a gait analysis system including a sensing unit, a storing unit storing a plurality of computing programs, and a processing unit electrically connected to the sensing unit and the storing unit, and comprises steps of: sensing a gait by the sensing unit to output a sensing signal, wherein a gait cycle includes a stance phase, a push-off phase, a swing phase and a heel-strike phase; obtaining a signal vector magnitude (SVM) and a signal magnitude subtraction (SMS) by the processing unit according to the sensing signal; identifying the stance phase, push-off phase, swing phase and heel-strike phase according to the SVM and SMS, wherein the push-off phase, swing phase and heel-strike phase are determined according to a dynamic threshold; and implementing a classification of the gait according to the stance phase, push-off phase, swing phase and heel-strike phase.

To achieve the above objective, a gait analysis system according to the invention comprises a sensing unit, a storing unit and a processing unit. The sensing unit senses a gait to output a sensing signal, and a gait cycle includes a stance phase, a push-off phase, a swing phase and a heel-strike phase. The storing unit stores a plurality of computing programs. The processing unit is electrically connected to the sensing unit and the storing unit, obtains a signal vector magnitude (SVM) and a signal magnitude subtraction (SMS) according to the sensing signal, and identifies the stance phase, push-off phase, swing phase and heel-strike phase according to the SVM and SMS for implementing a classification of the gait. The push-off phase, swing phase and heel-strike phase are determined according to a dynamic threshold.

In one embodiment, the processing unit obtains the SVM through the computation of an SVM computing program and obtains the SMS through the computation of an SMS computing program.

In one embodiment, the computation of the SVM computing program is implemented according to a first directional component, a second directional component and a third directional component of the sensing signal, and the computation of the SMS computing program is implemented according to the SVM and the second directional component.

In one embodiment, the processing unit computes according to the SMS through a standard deviation computing program, and the standard deviation computing program computes a standard deviation according to the SMS and identifies the stance phase of the SMS according to the SMS, standard deviation and a time threshold.

In one embodiment, a duration of the stance phase is greater than the time threshold.

In one embodiment, the initial value of the dynamic threshold is obtained according to the stance phase.

In one embodiment, the processing unit obtains the dynamic threshold through the computation of a dynamic threshold computing program, and the dynamic threshold computing program determines the dynamic threshold according to the SVM at different time points.

In one embodiment, if the SVM at a second time point is greater than or equal to the dynamic threshold at a first time point, the dynamic threshold at the second time point doesn't change.

In one embodiment, if the SVM at the second time point is less than the dynamic threshold at the first time point, the dynamic threshold at the second time point changes.

In one embodiment, the processing unit obtains the proportion of each of the stance phase, push-off phase, swing phase and heel-strike phase through the computation of a time computing program.

In one embodiment, if the sum of the durations of the push-off and swing phases is less than or equal to the duration of the heel-strike phase, the gait cycle is determined as a “downstairs-state”, and if the duration of the push-off phase is greater than the duration of the heel-strike phase, the gait cycle is determined as an “upstairs-state”.

In one embodiment, the gait analysis method further comprises a step of: computing a number of steps, step velocity, step length and distance of the whole gait by the processing unit according to the stance phase, push-off phase, swing phase and heel-strike phase.

As mentioned above, in the gait analysis method and gait analysis system of the invention, the sensing unit senses the gait to output the sensing signal, and then the processing unit obtains the SVM and SMS according to the sensing signal. Moreover, the stance phase, push-off phase, swing phase and heel-strike phase are identified according to the SVM and SMS, and the push-off phase, swing phase and heel-strike phase are determined according to the dynamic threshold. Subsequently, the classification of the gait is implemented according to the stance phase, push-off phase, swing phase and heel-strike phase. Thereby, the gait of the testee is analyzed and identified so that the doctor can provide the medical and healthy instructions for the testee according to the result of the analysis and identification.

### BRIEF DESCRIPTION OF THE DRAWINGS

The invention will become more fully understood from the detailed description and accompanying drawings, which are given for illustration only, and thus are not limitative of the present invention, and wherein:

FIG. 1A is a flow chart of a gait analysis method according to a preferred embodiment of the invention;

FIG. 1B is a schematic diagram of a gait cycle;

FIG. 2 is a schematic block diagram of a gait analysis system according to a preferred embodiment of the invention;

FIGS. 3A to 3C are schematic diagrams of the waveforms of the SVM when the testee walks on the ground, upstairs and downstairs, respectively;

FIGS. 4A to 4C are schematic diagrams of the waveforms of the SMS when the testee walks on the ground, upstairs and downstairs, respectively;

FIGS. 5A and 5B are schematic diagrams of the waveforms of another SMS when the testee walks;

FIG. 6 is a schematic diagram showing the signal waveform of a gait cycle;

FIGS. 7A to 7C are schematic diagrams showing the SVM and its corresponding dynamic threshold when the testee walks on the ground, upstairs and downstairs, respectively;

FIG. 8 is a judgment flowchart of the gait classification of the invention; and

FIG. 9 is another flow chart of a gait analysis method according to a preferred embodiment of the invention.

### DETAILED DESCRIPTION OF THE INVENTION

The present invention will be apparent from the following detailed description, which proceeds with reference to the accompanying drawings, wherein the same references relate to the same elements.

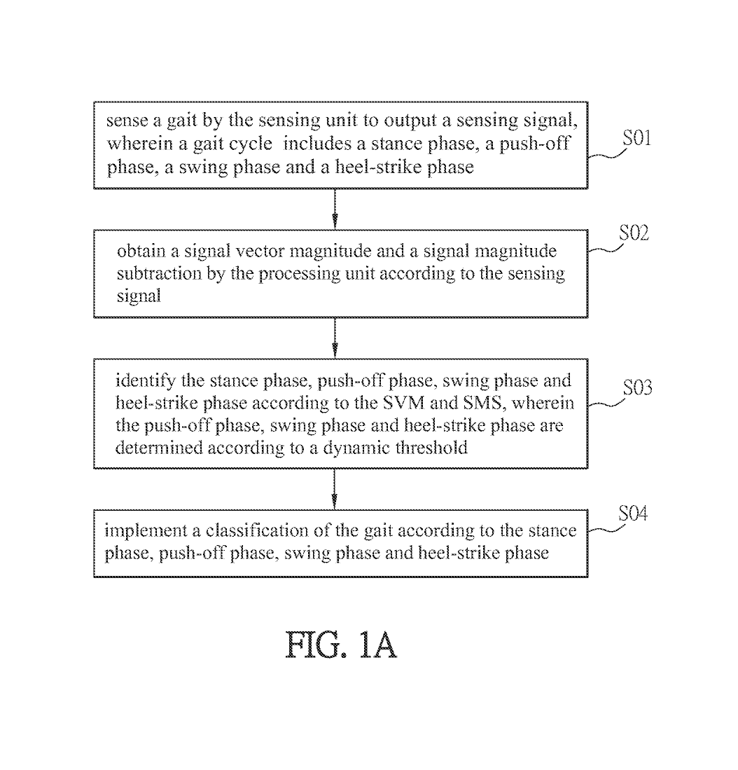

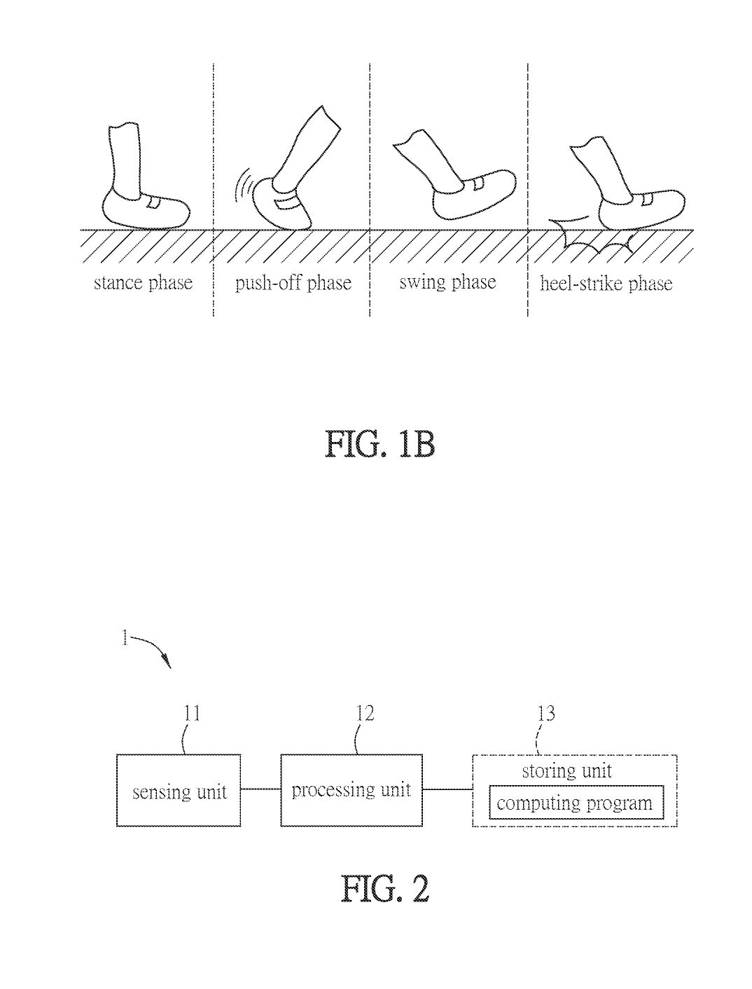

FIG. 1A is a flow chart of a gait analysis method according to a preferred embodiment of the invention, FIG. 1B is a schematic diagram of a gait cycle, and FIG. 2 is a schematic block diagram of a gait analysis system 1 according to a preferred embodiment of the invention.

The gait analysis method of this embodiment is implemented by the gait analysis system 1. As shown in FIG. 2, the gait analysis system 1 includes a sensing unit 11, a processing unit 12 and a storing unit 13. The processing unit 12 is electrically connected to the sensing unit 11 and the storing unit 13. The storing unit 13 stores a plurality of computing programs. As shown in FIG. 1A, the gait analysis method includes the steps S01 to S04.

The step S01 is to sense a gait by the sensing unit 11 to output a sensing signal, wherein a gait cycle (i.e. a complete step) includes a stance phase, a push-off phase, a swing phase and a heel-strike phase as shown in FIG. 1B. The sensing unit 11 of this embodiment is a wearable type, which is a triaxial accelerometer or gyroscope for example. Herein, the sensing unit 11 is a triaxial accelerometer as an example worn on the ankle of the testee. Accordingly, the sensing signal is a tri-directional acceleration signal (including a first directional component, a second directional and a third directional component, not shown). The gait referred to in the step S01 can be a step or a plurality of steps, and includes at least a complete gait cycle.

A gait cycle includes the stance phase, push-off phase, swing phase and heel-strike phase. In other words, when the testee wearing the sensing unit 11 walks for a distance, the sensing signal will be the tri-directional acceleration signal obtained within the distance. Herein, the “walking” may mean the testee wearing the sensing unit 11 walks on the ground, upstairs or downstairs. Before implementing the step S02, the processing unit 12 needs to do a pre-process to the sensing signal for reducing the influence of the baseline drift and high-frequency noise on the following gait analysis.

The step S02 is to obtain a signal vector magnitude (SVM) and a signal magnitude subtraction (SMS) by the processing unit 12 according to the sensing signal. The processing unit 12 obtains the SVM through the computation of an SVM computing program stored in the storing unit 13. Herein, the computation of the SVM computing program is implemented according to the first, second and third directional components, and the SVM is obtained in conformity to the equation as follows:

SVM(n)=\sqrt{aₓ²(n)+a_{y}²(n)+a_{z}²(n)}

In the above equation, aₓ, a_{y} and a_{z} denote the first, second and third directional components of the sensing signal, respectively, and n denotes the sampling point.

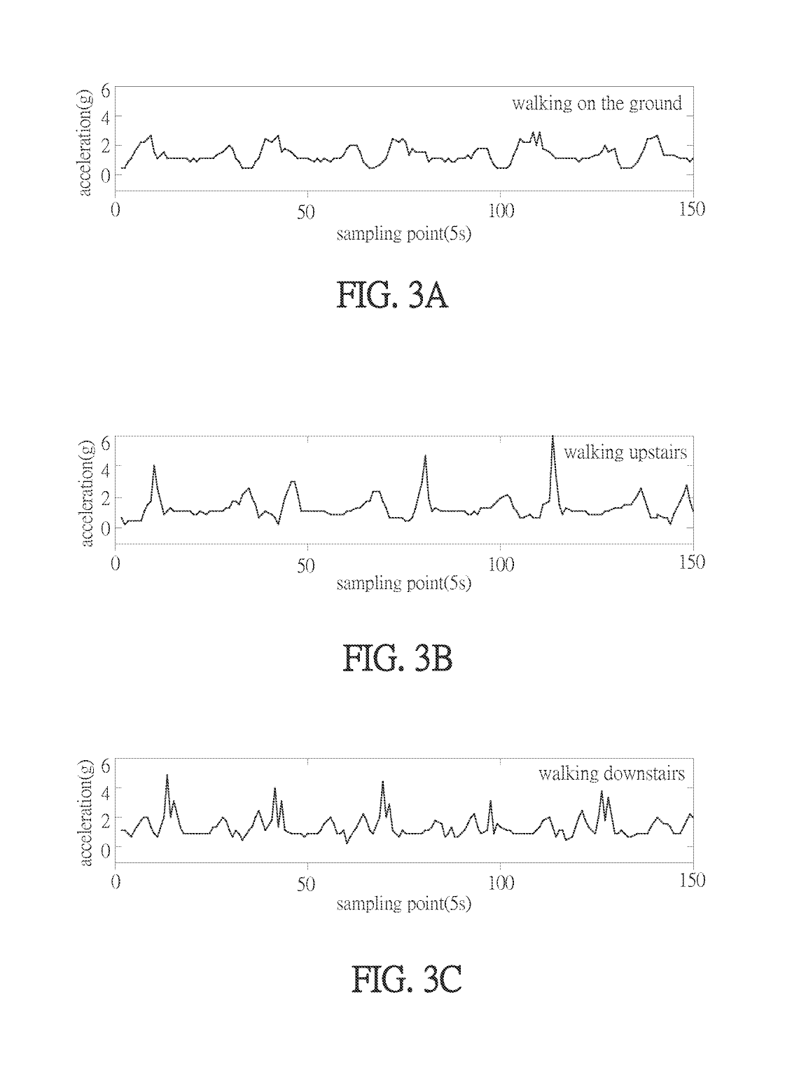

FIGS. 3A to 3C are schematic diagrams of the waveforms of the SVM obtained by the computation when the testee walks on the ground, upstairs and downstairs, respectively. The sampling duration is 5 seconds, and the sampling rate is 30 per second, so there are totally 150 sampling points on the abscissa. The ordinate shows the acceleration (g). Accordingly, each of FIGS. 3A to 3C shows a plurality of gait cycles. The above sampling duration, sampling rate and sampling points are just for example but not for limiting the scope of the invention. That is, they can be changed according to the requirements.

After obtaining the SVM, the processing unit 12 can further obtain the SMS through the computation of an SMS computing program stored in the storing unit 13. The computation of the SMS computing program is implemented according to the SVM and the second directional component a_{y} to obtain the SMS, as the following equation:

SMS(n)=SVM(n)−a_{y}(n)

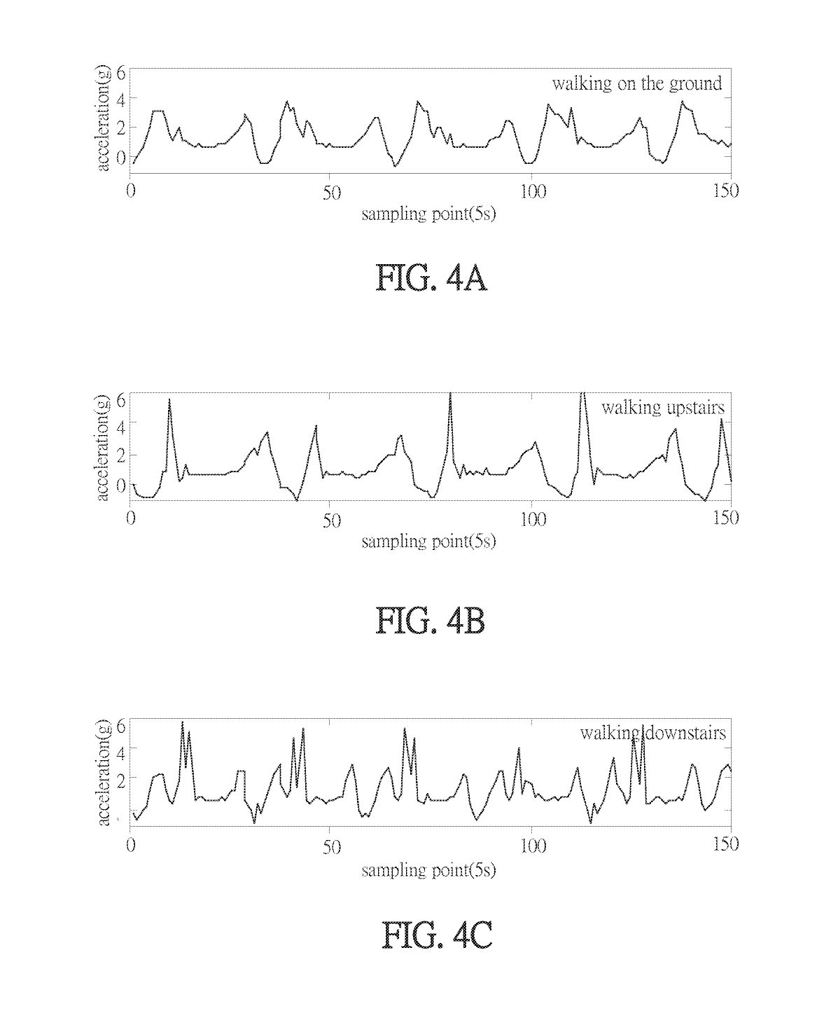

In the above equation, a_{y} denotes the second directional component of the sensing signal, and the second direction is the direction of gravity. In other words, the SMS as shown in FIGS. 4A to 4C can be obtained by subtracting the influence of gravity (1 g) from the SVM as shown in FIGS. 3A to 3C, respectively.

The step S03 is to identify the stance phase, push-off phase, swing phase and heel-strike phase according to the SVM and SMS, wherein the push-off phase, swing phase and heel-strike phase are determined according to a dynamic threshold DT. Herein at first, the processing unit 12 identifies the stance phase according to the SMS. In the step S03, the processing unit 12 computes according to the SMS through a standard deviation computing program stored in the storing unit 13 to obtain the stance phase of every gait cycle. Herein, the standard deviation computing program computes a standard deviation according to the SMS, and then identifies the stance phase of every gait cycle of the SMS according to the SMS, standard deviation and a time threshold STₘᵢₙ.

In detail, since there is no vertical movement of the foot of the testee during the stance phase of every gait cycle, the acceleration of the stance phase is relatively stable. Accordingly, the extraordinarily high and low signals of the SMS (caused by the counteraction of the ground) need to be eliminated first for obtaining the stance phase of the gait cycle. In this embodiment, the processing unit 12 first computes the standard deviation of the SMS to obtain an upper bound TH1u and a lower bound TH1d, and then eliminates the signals above the upper bound TH1u and below the lower bound TH1d from the SMS, as the following equations:

$$TH {1}_{u} = \frac{{\sum }_{n = 1}^{L} SMS ( n )}{L} + 2 \times \sqrt{\frac{1}{L - 1} {\sum }_{n = 1}^{L} {( SMS ( n ) - \overline{SMS ( n )} )}^{2}}$$

$$TH {1}_{d} = \frac{{\sum }_{n = 1}^{L} SMS ( n )}{L} - 2 \times \sqrt{\frac{1}{L - 1} {\sum }_{n = 1}^{L} {( SMS ( n ) - \overline{SMS ( n )} )}^{2}}$$

$${SMS}_{m} ( n ) = \{ \begin{matrix}SMS ( n ) , & if TH {1}_{d} < SMS ( n ) < TH {1}_{u} \\ 0 , & else\end{matrix}$$

In the above equations, L denotes the number of the signal points within the signal window, S̅M̅S̅(̅n̅)̅ denotes the average value of SMS(n), SMSₘ(n) denotes the signals of SMS(n) between the upper bound TH1u and the lower bound TH1d. The standard deviation is as follows:

$$\sqrt{\frac{1}{L - 1} {\sum }_{n = 1}^{L} {( SMS ( n ) - \overline{SMS ( n )} )}^{2}}$$

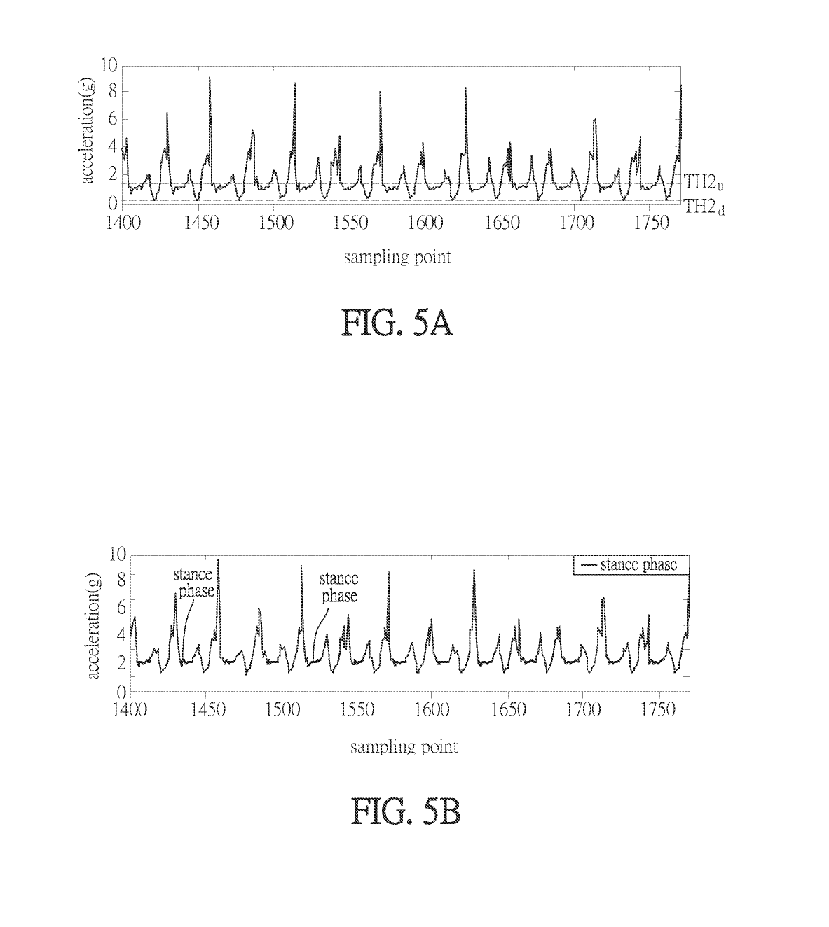

Next, according to the signal SMSₘ(n), another upper bound TH2u and another lower bound TH2d as shown in FIG. 5A are computed by the following equations:

$$TH {2}_{u} = \frac{{\sum }_{n = 1}^{l} {SMS}_{m} ( n )}{l} + \sqrt{\frac{1}{l - 1} {\sum }_{n = 1}^{{L}_{m}} {( {SMS}_{m} ( n ) - \overline{{SMS}_{m} ( n )} )}^{2}}$$

$$TH {2}_{d} = \frac{{\sum }_{n = 1}^{l} {SMS}_{m} ( n )}{l} - \sqrt{\frac{1}{l - 1} {\sum }_{n = 1}^{{L}_{m}} {( {SMS}_{m} ( n ) - \overline{{SMS}_{m} ( n )} )}^{2}}$$

In the above equations, “1” denotes the remaining sampling points after eliminating the extraordinarily high and low signals in the signal window, and S̅M̅S̅m̅(̅n̅)̅ denotes the average value of SMSₘ(n).

Then, as shown in FIG. 5A, the signals above the upper bound TH2u and lower bound TH2d are eliminated from the SMSₘ(n), so the remaining signals are within the upper bound TH2u and lower bound TH2d. Besides, since the signal of the stance phase usually remains for a while (that is, the foot will stand on the ground for a while), the stance phase will be identified also by ascertaining that a duration ΔT related to the signal is greater than the time threshold STₘᵢₙ, besides eliminating the signals above the upper bound TH2u and lower bound TH2d from the SMSₘ(n). That is, the signal of the stance phase is identified when the following equations are satisfied at the same time.

TH2_{d}<SMSₘ(n)<TH2ᵤΔT>STₘᵢₙ

By the above computation, the stance phase, shown by the solid line in FIG. 5B, of every gait cycle of the SMSₘ(n) can be identified. Since a complete step has a stance phase, the number of steps of the testee can be obtained when the number of the stance phase is known. As shown in FIG. 5B, there are 13 gait cycles with 13 stance phases in this gait, and thus the testee has 13 steps.

To be noted, the above computation algorithm is just for example. Different upper and lower bounds may be obtained by different computation algorithms for eliminating the extraordinarily high and low signals, and then the stance phase of every gait cycle can be identified according to SMSₘ(n). According to statistics, a complete step (a gait cycle) of an average person walking in a normal speed takes about 1.2˜1.3 seconds, and the stance phase takes 24.8% of the whole gait cycle. Therefore, in this embodiment, the time threshold STₘᵢₙ is set as 0.3 s (between 1.2×24.8% and 1.3×24.8%). In other words, in this embodiment, the duration of the stance phase needs to exceed 0.3 s to ascertain the stance phase. However, the time threshold STₘᵢₙ can be adjusted according to different testees. For example, when the testee is a person moving with difficulty, the time threshold STₘᵢₙ can be larger than 0.3 s. When the testee is an active young man, the time threshold STₘᵢₙ can be less than 0.3 s. To be noted, the signals in FIGS. 5A and 5B are just for the above computation but not following the signals in FIGS. 4A to 4C.

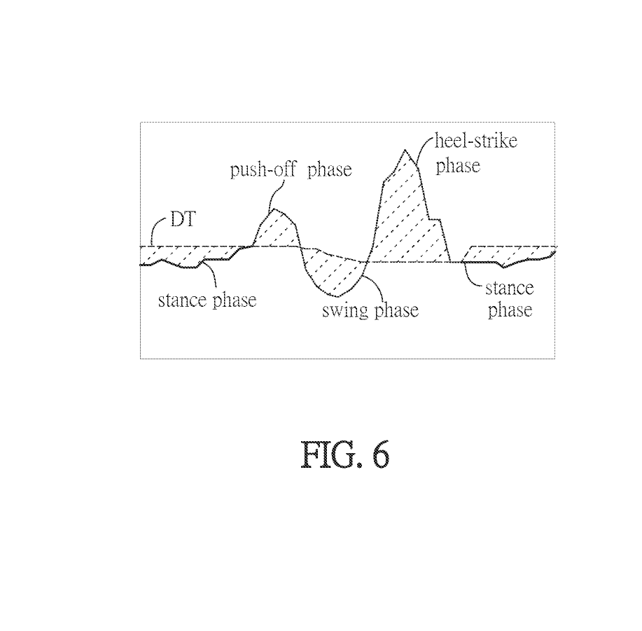

FIG. 6 is a schematic diagram showing the signal waveform of a gait cycle. Since the stance phase must be followed by the push-off phase, swing phase and heel-strike phase sequentially in a gait cycle, the signals after the signal of the stance phase can be known as corresponding to the push-off phase, swing phase and heel-strike phase, respectively.

Accordingly, after identifying the all stance phases in the gait, the processing unit 12 can identify the push-off phase, swing phase and heel-strike phase of every gait cycle according to the SVM, stance phase and dynamic threshold DT. The initial value of the dynamic threshold DT is determined according to the stance phase. Herein, the value of the last sampling point of the stance phase in a gait cycle is determined as the initial value of the dynamic threshold DT of the push-off phase in the same gait cycle. Besides, the processing unit 12 obtains the dynamic thresholds DT of every phase through the computation of a dynamic threshold computing program. The dynamic threshold computing program determines the dynamic thresholds DT according to the SVM at different time points, and determines the dynamic threshold DT of the next sampling point by the following equation:

$${DT}_{j} ( k ) = \{ \begin{matrix}{DT}_{j} ( k - 1 ) , & if SVM ( k ) \geq {DT}_{j} ( k - 1 ) \\ {DT}_{j} ( k - 1 ) + \frac{{SVM}_{j} ( k ) - {DT}_{j} ( k - 1 )}{S ( j )} , & otherwise\end{matrix}$$

In the above equation, SVM(k) denotes the value of the SVM at the k^{th} sampling point, DT(k) denotes the dynamic threshold DT at the k^{th} sampling point, and S(j) denotes the sum of the SVM of a certain gait cycle. Besides, if the SVM at the second time point k is greater than or equal to the dynamic threshold DT(k−1) (i.e. SVM(k)≧DT(k−1)) at the first time point k−1 (k−1 and k are consecutive sampling points), the dynamic threshold DT(k) at the second time point k will not change and will be the same as the dynamic threshold DT(k−1) at the first time point k−1 (i.e. DT(k)=DT(k−1)). Moreover, if the SVM at the second time point k is less than the dynamic threshold DT(k−1) at the first time point k−1 (i.e. SVM(k)<DT(k−1)), the dynamic threshold DT(k) at the second time point k will be obtained by the computation according to the above equation. So, this is why the dynamic threshold is called “dynamic”.

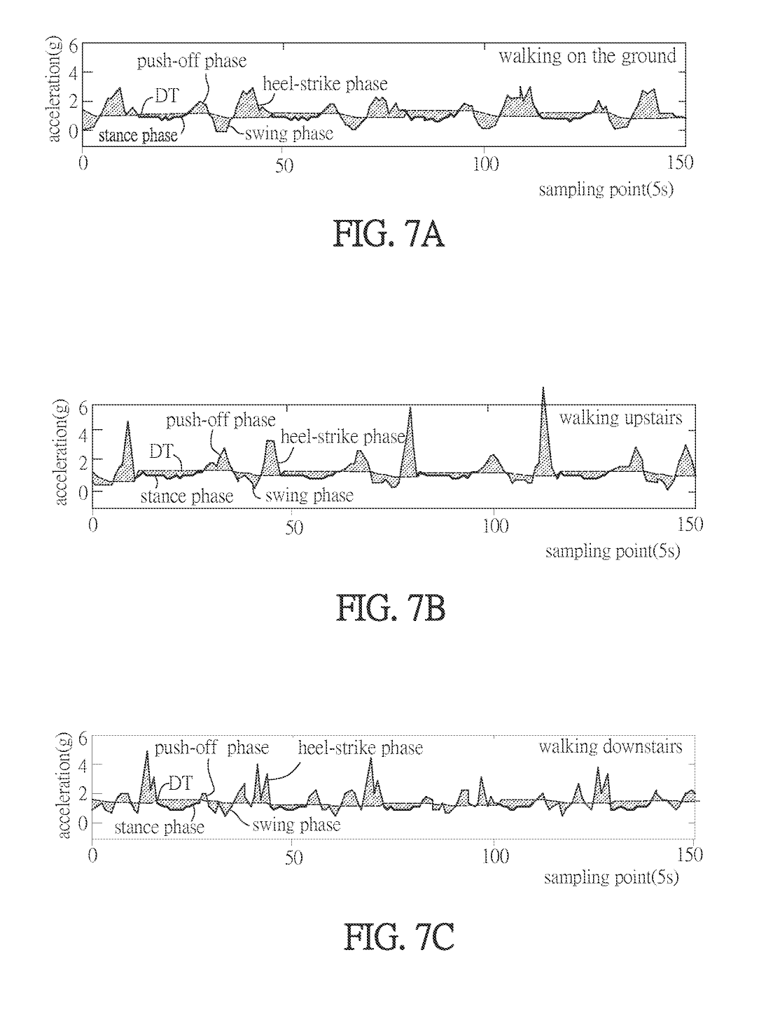

FIGS. 7A to 7C are schematic diagrams showing the SVM and its corresponding dynamic threshold DT when the testee walks on the ground, upstairs and downstairs, respectively. In FIGS. 7A to 7C, the solid line of the signal curve of SVM still denotes the stance phase.

By the above-mentioned judgment formula about the dynamic threshold DT, the dynamic thresholds DT corresponding to the stance phase, push-off phase, swing phase and heel-strike phase respectively in every gait cycle can be found out as shown in FIGS. 7A to 7C. In fact, according to the above judgment formula, the dynamic threshold DT will not change in the stance phase, push-off phase and heel-strike phase, and only in the swing phase the dynamic threshold DT will changes dynamically. Besides, the dynamic threshold DT doesn't change in the stance phase, and the signal of the last sampling point of the stance phase is used as the initial value of the dynamic threshold DT of the push-off phase. Furthermore, because the SVM is greater than the dynamic threshold DT in both of the push-off phase and heel-phase, the dynamic threshold DT of the push-off phase and heel-phase will not change. In addition, because the SVM is less than the dynamic threshold DT in the swing phase, the dynamic threshold DT of the swing phase will changes dynamically. Thereby, the stance phase, push-off phase, swing phase and heel-strike phase in every gait cycle can be identified.

The step S04 is to implement a classification of the gait according to the stance phase, push-off phase, swing phase and heel-strike phase. In the step S04, the processing unit 12 obtains the proportion of each of the stance phase, push-off phase, swing phase and heel-strike phase through the computation of a time computing program stored in the storing unit 13. In other words, the stance phase, push-off phase, swing phase and heel-strike phase of every gait cycle have been identified in the step S03, so the processing unit 12 can further obtain the proportion of each of the stance phase, push-off phase, swing phase and heel-strike phase in every gait cycle. Herein, the duration of the stance phase is defined as Ts, the duration of the push-off phase is defined as Tp, the duration of the swing phase is defined as Tw, and the duration of the heel-strike phase is defined as Th. Therefore, the duration of a gait cycle is equal to (Ts+Tp+Tw+Th). Besides, the proportion of the stance phase in the gait cycle is equal to Ts÷(Ts+Tp+Tw+Th)×100%, the proportion of the push-off phase in the gait cycle is equal to Tp÷(Ts+Tp+Tw+Th)×100%, the proportion of the swing phase in the gait cycle is equal to Tw÷(Ts+Tp+Tw+Th)×100%, and the proportion of the heel-strike phase in the gait cycle is equal to Th÷(Ts+Tp+Tw+Th)×100%.

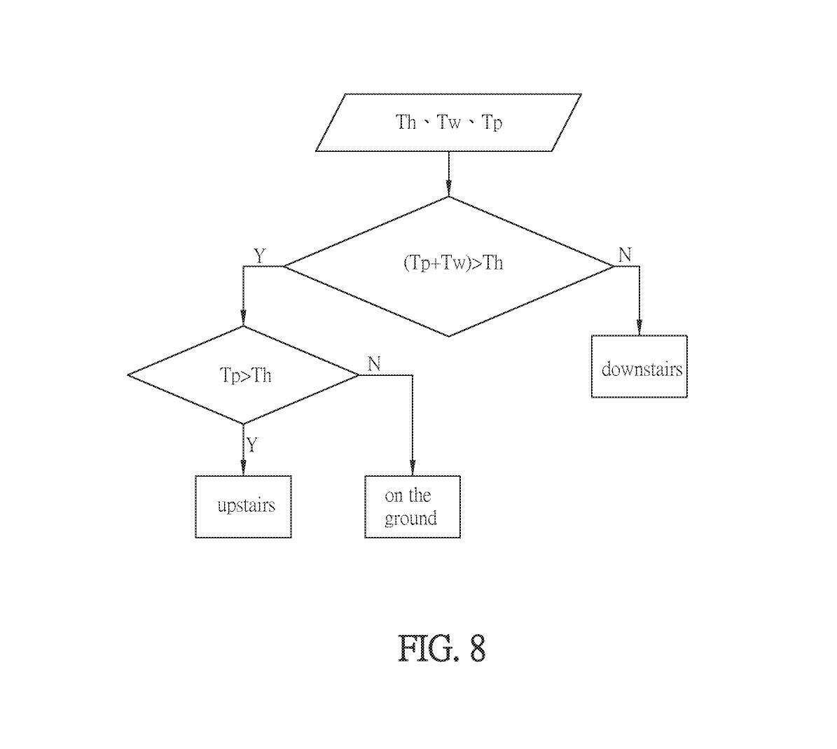

FIG. 8 is a judgment flowchart of the classification of the gait of the invention. The classification of the gait cycle is implemented by the duration of the heel-strike phase Th, the duration of the swing phase Tw and the duration of the push-off phase Tp. The processing unit 12 implements the classification through a classification computing program stored in the storing unit 13.

As shown in FIG. 8, if the sum of the durations of the push-off and swing phases (Tp+Tw) is less than or equal to the duration of the heel-strike phase Th, the gait cycle is determined as a “downstairs-state”. If the sum of the durations of the push-off and swing phases (Tp+Tw) is greater than the duration of the heel-strike phase Th and the duration of the push-off phase Tp is greater than the duration of the heel-strike phase Th, the gait cycle is determined as an “upstairs-state”. Besides, If the sum of the durations of the push-off and swing phases (Tp+Tw) is greater than the duration of the heel-strike phase Th and the duration of the push-off phase Tp is less than or equal to the duration of the heel-strike phase Th, the gait cycle is determined as a “ground-state”. By such kind of classification, every gait cycle of the whole gait of the testee is classified into one of the three states. Thereby, the doctor, therapist or testee can understand if the gait of the testee causes an excessively big burden to the body of the testee. For example, if a testee with bad knees is examined there is a larger proportion for the upstairs-state and/or downstairs-state in the gait cycle thereof, the doctor or therapist can make the medical and healthy instructions for the testee, such as cutting down the proportion of going upstairs and downstairs, or eating more proper food, for avoiding further harm to the knees.

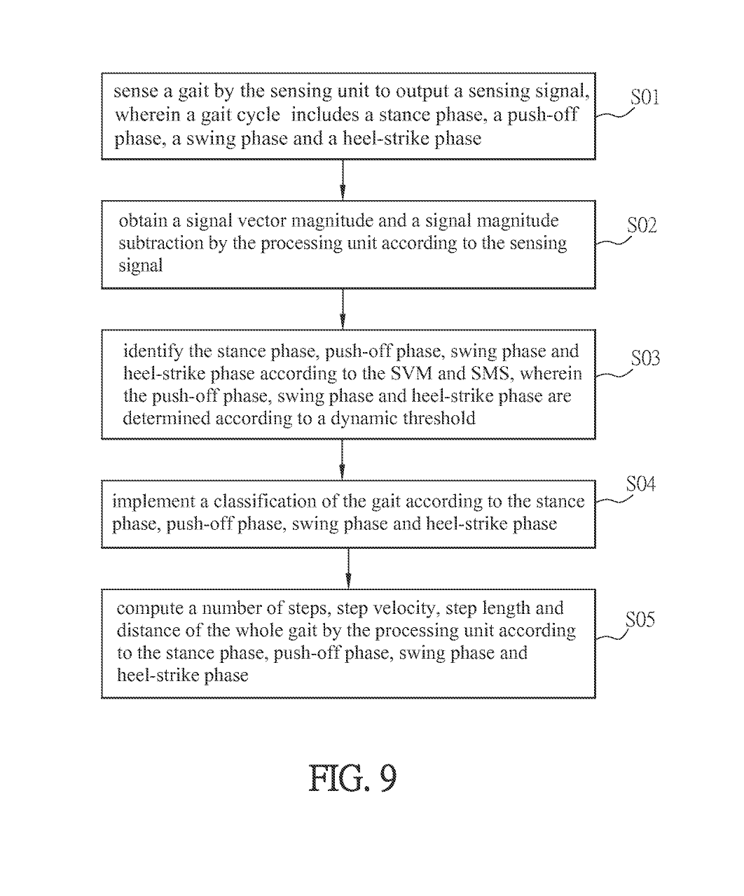

FIG. 9 is another flow chart of a gait analysis method according to a preferred embodiment of the invention.

Herein, besides the steps S01 to S04, the gait analysis method of this embodiment can further include a step S05, which is to compute a number of steps, step velocity, step length and distance of the whole gait by the processing unit 12 according to the stance phase, push-off phase, swing phase and heel-strike phase. Since the duration and proportion of each of the stance phase, push-off phase, swing phase and heel-strike phase in every gait cycle of the gait of the testee are obtained, a number of steps, step velocity, step length and distance of the whole gait can be all computed accordingly. In detail, the number of steps is the quantity of the gait cycles (or quantity of one of the stance phase, push-off phase, swing phase and heel-strike phase) of the whole gait. Moreover, the number of the gait cycles of the SVM is the number of steps, and the distance of the whole gait can be obtained by multiplying the number of the steps by the step length (Step_{length}). Besides, the step velocity (Step_{velocity}) and step length (Step_{length}) can be obtained by the regression analysis in cooperation with the following equations:

Step_{length}=70.9−36.1Step_{frequence}+52.0Step_{velocity}

Step_{velocity}=0.64+0.26Variance_{SVMxyz}+0.59Average_{Y}

In the above equations, Variance_{SVMxyz} is a variance of the SVM, Average_{Y} is the second directional component of the SVM, and the Step_{frequency} is the step frequency of the testee when walking. To be noted, the above equations about the step velocity (Step_{velocity}) and step length (Step_{length}) are just for example but not for limiting the scope of the invention.

In summary, in the gait analysis method and gait analysis system of the invention, the sensing unit senses the gait to output the sensing signal, and then the processing unit obtains the SVM and SMS according to the sensing signal. Moreover, the stance phase, push-off phase, swing phase and heel-strike phase are identified according to the SVM and SMS, and the push-off phase, swing phase and heel-strike phase are determined according to the dynamic threshold. Subsequently, the classification of the gait is implemented according to the stance phase, push-off phase, swing phase and heel-strike phase. Thereby, the gait of the testee is analyzed and identified so that the doctor can provide the medical and healthy instructions for the testee according to the result of the analysis and identification.

Although the invention has been described with reference to specific embodiments, this description is not meant to be construed in a limiting sense. Various modifications of the disclosed embodiments, as well as alternative embodiments, will be apparent to persons skilled in the art. It is, therefore, contemplated that the appended claims will cover all modifications that fall within the true scope of the invention.

What is claimed is:

1. A gait analysis method implemented by a gait analysis system including a sensing unit, a processing unit and a storing unit, wherein the processing unit is electrically connected to the sensing unit and the storing unit and the storing unit stores a plurality of computing programs, the gait analysis method comprising steps of:
sensing a gait by the sensing unit to output a sensing signal, wherein a gait cycle includes a stance phase, a push-off phase, a swing phase and a heel-strike phase;
obtaining a signal vector magnitude (SVM) and a signal magnitude subtraction (SMS) by the processing unit according to the sensing signal;
identifying the stance phase, push-off phase, swing phase and heel-strike phase according to the SVM and SMS, wherein the push-off phase, swing phase and heel-strike phase are determined according to a dynamic threshold; and
implementing a classification of the gait according to the stance phase, push-off phase, swing phase and heel-strike phase.

2. The gait analysis method as recited in claim 1, wherein in the step of obtaining the SVM and SMS, the processing unit obtains the SVM through the computation of an SVM computing program and obtains the SMS through the computation of an SMS computing program.

3. The gait analysis method as recited in claim 2, wherein the computation of the SVM computing program is implemented according to a first directional component, a second directional component and a third directional component of the sensing signal, and the computation of the SMS computing program is implemented according to the SVM and the second directional component.

4. The gait analysis method as recited in claim 1, wherein in the step of identifying the stance phase, push-off phase, swing phase and heel-strike phase, the processing unit computes according to the SMS through a standard deviation computing program, and the standard deviation computing program computes a standard deviation according to the SMS and identifies the stance phase of the SMS according to the SMS, standard deviation and a time threshold.

5. The gait analysis method as recited in claim 4 wherein a duration of the stance phase is greater than the time threshold.

6. The gait analysis method as recited in claim 4 wherein the initial value of the dynamic threshold is obtained according to the stance phase.

7. The gait analysis method as recited in claim 6 wherein the processing unit obtains the dynamic threshold through the computation of a dynamic threshold computing program, and the dynamic threshold computing program determines the dynamic threshold according to the SVM at different time points.

8. The gait analysis method as recited in claim 7, wherein if the SVM at a second time point is greater than or equal to the dynamic threshold at a first time point, the dynamic threshold at the second time point doesn't change.

9. The gait analysis method as recited in claim 8, wherein if the SVM at the second time point is less than the dynamic threshold at the first time point, the dynamic threshold at the second time point changes.

10. The gait analysis method as recited in claim 1, wherein in the step of the implementing a classification of the gait, the processing unit obtains the proportion of each of the stance phase, push-off phase, swing phase and heel-strike phase through the computation of a time computing program.

11. The gait analysis method as recited in claim 10, wherein if the sum of the durations of the push-off and swing phases is less than or equal to the duration of the heel-strike phase, the gait cycle is determined as a “downstairs-state”, and if the duration of the push-off phase is greater than the duration of the heel-strike phase, the gait cycle is determined as an “upstairs-state”.

12. The gait analysis method as recited in claim 1, further comprising a step of:
computing a number of steps, step velocity, step length and distance of the whole gait by the processing unit according to the stance phase, push-off phase, swing phase and heel-strike phase.

13. A gait analysis system, comprising:
a sensing unit sensing a gait to output a sensing signal, wherein a gait cycle includes a stance phase, a push-off phase, a swing phase and a heel-strike phase;
a storing unit storing a plurality of computing programs; and
a processing unit electrically connected to the sensing unit and the storing unit, obtaining a signal vector magnitude (SVM) and a signal magnitude subtraction (SMS) according to the sensing signal, and identifying the stance phase, push-off phase, swing phase and heel-strike phase according to the SVM and SMS for implementing a classification of the gait,
wherein the push-off phase, swing phase and heel-strike phase are determined according to a dynamic threshold.

14. The gait analysis system as recited in claim 13, wherein the processing unit obtains the SVM through the computation of an SVM computing program and obtains the SMS through the computation of an SMS computing program.

15. The gait analysis system as recited in claim 14, wherein the computation of the SVM computing program is implemented according to a first directional component, a second directional component and a third directional component of the sensing signal, and the computation of the SMS computing program is implemented according to the SVM and the second directional component.

16. The gait analysis system as recited in claim 13, wherein the processing unit computes according to the SMS through a standard deviation computing program, and the standard deviation computing program computes a standard deviation according to the SMS and identifies the stance phase of the SMS according to the SMS, standard deviation and a time threshold.

17. The gait analysis system as recited in claim 16, wherein a duration of the stance phase is greater than the time threshold.

18. The gait analysis system as recited in claim 13, wherein the processing unit obtains the dynamic threshold through the computation of a dynamic threshold computing program, and the dynamic threshold computing program determines the dynamic threshold according to the SVM at different time points.

19. The gait analysis system as recited in claim 18, wherein if the SVM at a second time point is less than the dynamic threshold at a first time point, the dynamic threshold at the second time point changes.

20. The gait analysis system as recited in claim 13, wherein the processing unit obtains the proportion of each of the stance phase, push-off phase, swing phase and heel-strike phase through the computation of a time computing program, and computes a number of steps, step velocity, step length and distance of the whole gait according to the stance phase, push-off phase, swing phase and heel-strike phase.
